(19) United States
(12) Patent Application Publication
(10) Pub. No.: US 2014/0330104 A1
(43) Pub. Date: Nov. 6, 2014

(54) NON-CONTACT TONOMETER

(71) Applicant: CANON KABUSHIKI KAISHA, Tokyo (JP)

(72) Inventors: Koichi Ohta, Yokohama-shi (JP); Yasuhiro Dobashi, Matsudo-shi (JP)

(73) Assignee: CANON KABUSHIKI KAISHA, Tokyo (JP)

(21) Appl. No.: 14/257,137

(22) Filed: Apr. 21, 2014

(30) Foreign Application Priority Data
May 1, 2013 (JP) 2013-096401

(51) Int. Cl. A61B 3/16

(52) U.S. Cl. CPC A61B 3/165
USPC 600/401

(57) ABSTRACT

Provided is a non-contact tonometer capable of inhibiting unnecessary air discharge against an eye to be inspected. The non-contact tonometer includes: a corneal shape deforming unit for pressurizing air in a cylinder using a piston provided in the cylinder and puffing the air through an opening in the cylinder toward a cornea of an eye to be inspected so as to deform the cornea; a piston control unit for controlling operation of the piston; and an eye pressure measurement unit for detecting a state of deformation of the cornea so as to measure an eye pressure of the eye to be inspected. The piston includes: an air ejecting portion provided on the opening side in the cylinder; and a piston drive portion connected to the piston control unit independently of the air ejecting portion.

### BACKGROUND OF THE INVENTION

1. Field of the Invention

The present invention relates to a non-contact tonometer for calculating an eye pressure value based on a corneal shape deformation signal from an optical detection unit when air is puffed against an eye to be inspected to deform a cornea of the eye.

2. Description of the Related Art

Non-contact tonometers are typified by an air-puff tonometer developed by Bernard Grolman. In the tonometer, air is puffed against an eye to be inspected from a nozzle which is about 11 mm away from a cornea of the eye. Then, corneal applanation is optically detected, and a time period until the applanation is calibrated by a Goldman contact tonometer, to thereby calculate an eye pressure value.

In many of such air-puff tonometers, there is used such, a system that air in a cylinder connected to an air discharge nozzle portion is pressurized through movement of a piston in the cylinder to discharge air from the nozzle. Further, as a drive system of the piston, a solenoid is typically used from the viewpoint of its high initial torque and simple structure.

Further, a non-contact tonometer is required to have a wide measurement range adaptable to a low eye pressure and a high eye pressure clue to a disease such as glaucoma. In order to measure a high eye pressure of an eye to be inspected, it is necessary to discharge enough air for the eye to be inspected, and thus, a volume of the cylinder is designed based on a high eye pressure.

Therefore, for an eye to be inspected having a low eye pressure, a magnitude of a drive current supplied to the solenoid and a drive time period of the solenoid are changed in accordance with the eye pressure value of the eye to be inspected, to thereby adjust an amount of the air to be puffed.

However, while a system using a solenoid is low in cost and simple in structure, some demerits thereof are known. A solenoid is simple in structure and includes only a coil and a permanent magnet, and an actuation direction thereof is limited to only one direction. Thus, a return system such as a return spring is required to be additionally used.

Normally, an actuation force of a solenoid is sufficiently stronger than that of the return spring, and, once the solenoid is energized to drive the piston, an inertia force due to a mass of the piston itself is exerted even after a current therethrough is stopped. Therefore, it is difficult to stop the piston at a desired position.

In particular, when a low eye pressure of an eye to be inspected is measured, the amount of air necessary for the applanation thereof is small, and thus, it is necessary to stop the piston at quite an early stage in a drive range of the piston in the cylinder, but due to the inertia force of the piston, air unnecessary for the measurement is discharged against the eye to be inspected, which is a cause of discomfort of a subject.

In order to solve the above-mentioned problem, 1) Japanese Patent Application Laid-Open No. H09-201335 discloses an invention which reduces, by increasing a drive voltage to foe applied to the solenoid for driving the piston at a low rate, the amount of movement of the piston due to the inertia force after a piston drive current is shut off.

Further, 2) Japanese Patent Application Laid-Open No. 2002-34927 discloses a system for causing air to escape using a solenoid valve for the purpose of preventing pressurized air in the cylinder from being discharged against the eye to be inspected. According to the invention disclosed in Japanese Patent Application Laid-Open No. 2002-34927, unnecessary air discharge against the eye to be inspected is reduced by, in addition to the system for causing air in the cylinder to escape using the solenoid valve, opening the solenoid valve at an appropriate timing through a prediction on a timing for opening the solenoid valve based on a first measurement in consideration of a response delay of the solenoid valve.

However, even a circuit in which the voltage to be applied is gradually increased as in Japanese Patent Application Laid-Open No. H09-201335 has problems in that discharge of air due to the inertia force of the piston cannot be prevented and the control circuit is complicated when the voltage to be applied is set variable.

Further, even if a sudden stop of the piston can be made by a certain control system, pressurized air in the cylinder has a pressure which is higher than atmospheric pressure, and thus, the air leaks from the discharge nozzle. Therefore, the basic problem in that air which is a cause of discomfort of a subject is discharged is not solved.

Further, the method of causing pressurized air in the cylinder to escape by opening the solenoid valve as disclosed in Japanese Patent Application Laid-Open No. 2002-34927 is theoretically effective. However, in order to instantaneously release pressurized air in the cylinder, it is necessary that a release port in the solenoid valve be set sufficiently large with respect to the nozzle, which requires a large solenoid valve. A large solenoid valve is high in cost and is difficult to mount in a limited space of the tonometer, which raises a hurdle for the adoption thereof.

### SUMMARY OF THE INVENTION

The present invention is to provide a non-contact tonometer capable of solving the above-mentioned problems and inhibiting unnecessary air discharge against an eye to be inspected at a low cost with a simple structure.

According to one embodiment of the present invention, there is provided a non-contact tonometer, including; a corneal shape deforming unit for pressurizing air in a cylinder using a piston provided in the cylinder and puffing the air through an opening in the cylinder toward a cornea of an eye to be inspected so as to deform the cornea; a piston control unit for controlling operation of the piston; and an eye pressure measurement unit for detecting a state of deformation of the cornea so as to measure an eye pressure of the eye to be inspected, the piston including; an air ejecting portion provided on the opening side in the cylinder; and a piston drive portion connected to the piston control unit independently of the air ejecting portion.

The non-contact tonometer according one embodiment of the present invention can discharge an optimum amount of air.

Further features of the present invention will become apparent from the following description, of exemplary embodiment 1 with reference to the attached drawings.

### BRIEF DESCRIPTION OF THE DRAWINGS

FIG. 1 is an outer appearance view of a non-contact tonometer.

FIG. 2 is an arrangement view of an optical system of a measurement portion,

FIG. 3 is a system block diagram according to a first embodiment of the present invention.

FIGS. 4A, 4B and 4C are explanatory diagrams of piston positions in a related-art control method.

FIG. 5 is a graph showing a relationship between a corneal shape deformation signal and a pressure signal in the related-art control method.

FIG. 6 is an explanatory diagram, of a separable structure of a piston portion according to the first embodiment of the present invention.

FIGS. 7A, 7B, 7C and 7D are explanatory diagrams of piston operation in a control method according to the first embodiment of the present invention.

FIG. 8 is a graph showing a relationship between a corneal shape deformation signal and a pressure signal in the control method according to the first embodiment of the present invention.

FIG. 9 is a flow chart illustrating the first embodiment of the present invention.

### DESCRIPTION OF THE EMBODIMENTS

In a non-contact tonometer according to the present invention, a piston in a cylinder has a separable structure including an air ejecting portion and a piston drive portion. Further, an initial position of the ejecting portion can be changed in accordance with an eye pressure value. The above-mentioned structure enables discharge of an optimum amount of air. Further, by separating the air ejecting portion, and the piston drive portion, suction of tears or the like after the air discharge can be avoided. Further, the structure can be formed only by adding a piston position detection system and an electromagnet to a related-art tonometer, and thus, a low-cost and small tonometer can be provided.

Now, an embodiment of the present invention is described in detail with reference to the attached drawings.

### First Embodiment

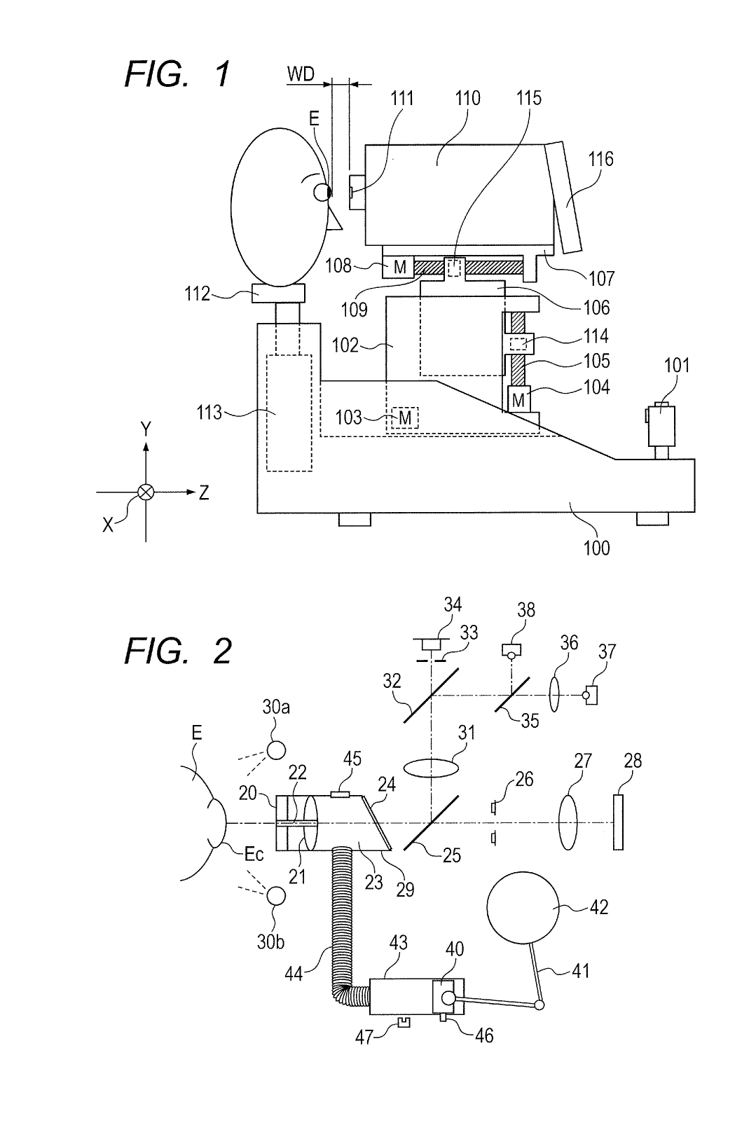

FIG. 1 is a schematic structural view of a non-contact tonometer according to a first embodiment of the present invention.

A frame 102 can be moved with respect to a base 100 in a lateral direction (hereinafter referred to as an X axis direction which is perpendicular to a plane of the drawing). A drive system in the X axis direction includes an X axis drive motor 103 fixed onto the base 100, a feed screw (not shown) coupled to a motor output shaft; and a nut (not shown) which can be moved in the X axis direction on the feed screw and is fixed to the frame 102. Through rotation of the motor 103, the frame 102 is moved in the X axis direction through intermediation of the feed screw and the nut.

A frame 106 can be moved with respect to the frame 102 in a vertical direction (hereinafter referred to as a Y axis direction). A drive system in the Y axis direction includes a Y axis drive motor 104 fixed onto the frame 102, a feed screw 105 coupled to a motor output shaft, and a nut 114 which can be moved in the Y axis direction on the feed screw and is fixed to the frame 106. Through rotation of the motor 104, the frame 106 is moved in the Y axis direction through intermediation of the feed screw and the nut.

A frame 107 can be moved with respect to the frame 106 in a fore and aft direction (hereinafter referred to as a Z axis direction). A drive system in the Z axis direction includes a Z axis drive motor 108 fixed onto the frame 107, a feed screw 109 coupled to a motor output shaft, and a nut 115 which can be moved in the Z axis direction on the feed screw and is fixed to the frame 106. Through rotation of the motor 108, the frame 107 is moved in the Z axis direction through intermediation of the feed screw and the nut.

Movement of the frame 102 in the X axis direction corresponds to lateral movement of the tonometer with respect to a subject, movement of the frame 106 in the Y axis direction corresponds to vertical movement of the tonometer with respect to the subject, and movement of the frame 107 in the Z axis direction corresponds to fore and aft movement of the tonometer toward and away from the subject.

A measurement portion 110 for measurement is fixed onto the frame 107. A nozzle 111 for discharging air necessary for measuring an eye pressure is provided at an end of the measurement portion 110 on the subject side. An LCD monitor 116 as a display member for observing an eye E to be inspected is provided at an end of the measurement portion 110 on an examiner side.

The base 100 includes a joystick 101 as an operation member for positioning the measurement portion 110 with respect to the eye E to be inspected.

When the eye pressure is measured; the subject rests his/her chin on a chin rest 112 and presses his/her forehead against a forehead rest portion of a face rest frame (not shown) which is fixed to the base 100, thereby enabling fixation, of the position of the eye to be inspected. The position of the chin rest 112 can be adjusted in the Y axis direction by a chin rest motor 113 in accordance with a size of a face of the subject.

FIG. 2 is a structural view of an optical system in the measurement portion 110 in a related-art structure. A nozzle 22 is provided on a central axis of a plane parallel glass plate 20 and of an objective lens 21 so as to be opposed to a cornea Ec of the eye E to be inspected, and an air chamber 23, an observation window 24, a dichroic mirror 25, a prism aperture 26, an imaging lens 27, and a CCD 28 are arranged in this order at the back thereof. These components form an optical path for receiving light and an optical path for detecting alignment of an observation optical system for the eye E to be inspected.

The plane parallel glass plate 20 and the objective lens 21 are supported by an objective lens barrel 29, and anterior ocular segment illumination light sources 30a and 30b for illuminating the eye E to be inspected are arranged outside thereof.

Note that, for the sake of simplicity, the anterior ocular segment illumination light sources 30a and 30b are illustrated in an upper part and a lower part of the figure, respectively, but actually, the anterior ocular segment illumination light sources 30a and 30b are arranged so as to be opposed to each other with respect to an optical axis in a direction perpendicular to a plane of the drawing.

A relay 31, a half mirror 32, an aperture 33, and a light receiving element 34 are arranged in a reflection direction of the dichroic mirror 25. Note that, the aperture 33 is provided at a position conjugate to a corneal reflection image of a measurement light source 37 described below when the cornea is deformed into a predetermined shape, and constructs, together with the light receiving element 34, a deformation detection light receiving optical system which is used for detecting an amount of deformation when the cornea Ec is deformed in a direction of the visual axis.

The relay lens 31 is designed so as to form a corneal reflection image which is substantially the same size as the aperture 33 when the cornea Ec is deformed into a predetermined shape. A half mirror 35, a projection lens 36, and the measurement light, source 37 which is a near infrared LED for emitting light having a wavelength of invisible light used both for measurement, and for alignment with respect to the eye E to be inspected are arranged in a direction of light reflected by the half mirror 32, and a fixation target light source 38 as a fixation target of the subject which is an LED is provided in a direction of light reflected by the half mirror 35.

A pressure sensor 45 for monitoring an internal pressure of the air chamber and a transfer tube 44 for transferring pressurized air from a cylinder 43 are connected to the inside of the air chamber 23. The transfer tube may be a tube of any type including an accordion tube as illustrated in FIG. 2, and a metal tube. Further, the cylinder 43 may be provided so as to be directly connected to the air chamber 23 without using the transfer tube 44, A piston 40 fits into the cylinder 43, The piston 40 is driven by a solenoid 42.

Rotational motion of the solenoid 42 is converted, into linear motion of the piston 40 through a drive lever 41 which is connected to the solenoid 42 and to the piston 40. In this system, the piston 40 moves in the cylinder 43 ids high speed to send pressurized air in the cylinder 43 into the air chamber 23 and to discharge air through the nozzle 22 against the eye E to be inspected.

A structure including the cylinder 43, the piston 40, and the like forms an exemplary corneal shape deforming unit for pressurizing air in the cylinder by the piston, which is provided in the cylinder and operates from an operation start position, and for puffing the pressurized air from inside the cylinder toward the cornea of the eye to be inspected to deform the cornea.

Further, a detection sensor dog 46 for detecting a piston position is connected to the piston 40, The sensor dog 46 and a detection switch 47 enable detection of the position of the piston 40.

Further, it is not necessary to provide the sensor dog 45 and the detection switch 47 in proximity to the cylinder 43 as illustrated in FIG. 2, The sensor dog 46 and the detection switch 47 may be provided in proximity to the solenoid 42 and the position of the piston 40 may be detected based on a rotational angle of the solenoid 42. This structure is an exemplary piston position detecting unit for detecting the position of the piston.

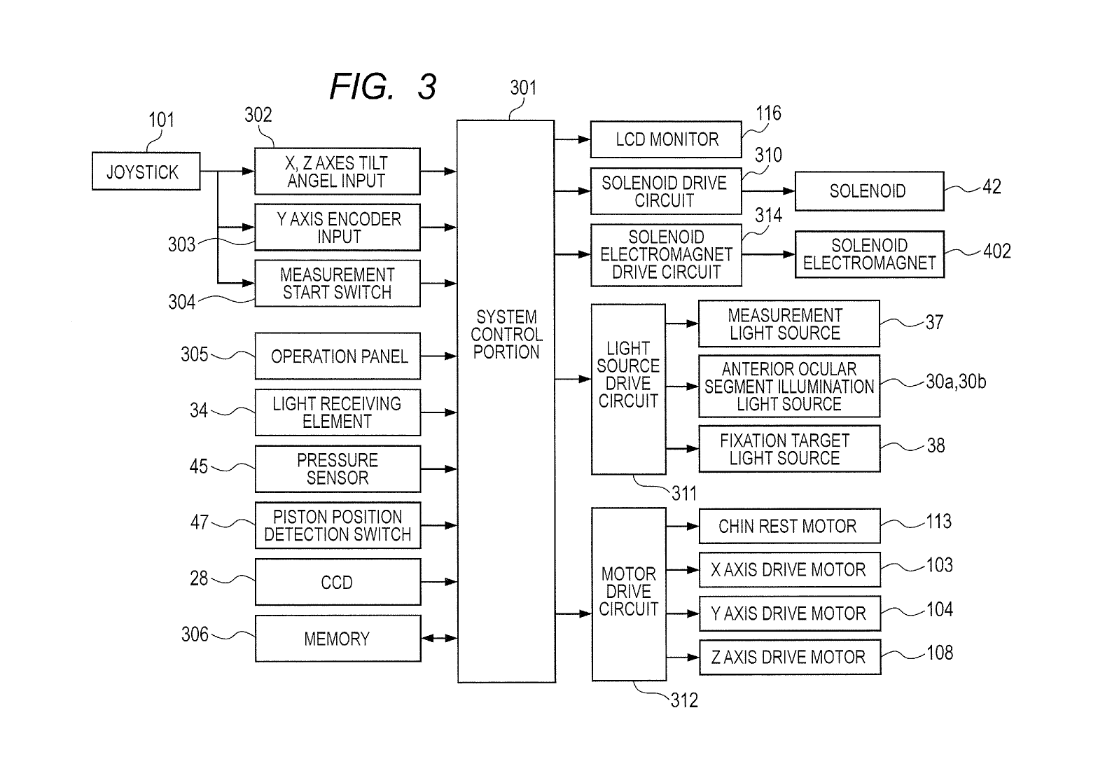

FIG. 3 is a system block diagram. A system control portion 301 for controlling an entire system includes a program storage portion, a data storage portion for storing data for correcting an eye pressure value, an input/output control portion for controlling input from and output to various kinds of devices, and a computation processing portion for computing data obtained, from the various kinds of devices.

Input from an X, Z axes tilt angle input 302 in the case of tilt in the fore and aft direction and the lateral direction, input from a Y axis encoder input 303 in the case of rotation, and input from a measurement start button 304 in the case of depressing the measurement start button are connected from the joystick 101 to the system control portion 301 for the purpose of positioning the measurement portion 110 with respect to the eye E to be inspected and starting the measurement.

Further, a print button, a chin rest up/down button, and the like are arranged on an operation panel 305 (not shown) on the base 100, and, when any button is depressed for input, a signal is sent to the system control portion. An anterior ocular segment image of the eye E to be inspected, which is picked up by the CCD 28, is stored in a memory 306.

A pupil of the eye E to be inspected, and the corneal reflection image are extracted from the image stored in the memory 306, and alignment is detected. Further, the anterior ocular segment image of the eye E to be inspected, which is picked up by the CCD 28, is combined with character data and graphic data, and the anterior ocular segment image, the measured value, and the like are displayed on the LCD monitor 116.

A corneal shape deformation signal received by the light receiving element 34 and a signal from the pressure sensor 45 provided in the air chamber 23 are stored in the memory 306. A structure including the light receiving element 34 and the like for detecting a corneal shape deformation signal which indicates the state of corneal shape deformation to measure the eye pressure of the eye to be inspected is an exemplary structure which functions as an eye pressure measurement unit according to the present invention.

The X axis drive motor 103, the Y axis drive motor 104, the Z axis drive motor 108, and the chin rest motor 113 are driven by a command from the system control portion 301 via a motor drive circuit 312, The measurement light source 37, the anterior ocular segment illumination light sources 30a and 30b, and the fixation target light source 38 control ON/OFF and change in light amount by a command from the system control portion 301 via a light source drive circuit 311.

The solenoid 42 is controlled by a signal from the system control portion 301, and change in drive current and ON/OFF of voltage application are performed via a solenoid drive circuit 310.

In this case, a rotary solenoid is used as the solenoid 42. When a voltage is applied to the rotary solenoid, a movable pin moves in a coil formed by winding a copper wire, and the linear motion is converted into rotational motion by a mechanism including a bearing and the like. A rotational torque of the rotary solenoid is limited to one direction, and thus, the rotary solenoid in this structure returns to an initial position thereof by a built-in coil spring.

When a value of a drive current flowing through the solenoid 42 is set to be high under the control of the solenoid drive circuit 310, a high torque is produced in the solenoid 42 to enable high-speed, rotation of the solenoid. Further, as described above, the rotary solenoid includes the built-in coil spring for returning the rotary solenoid to the initial position.

Therefore, by causing a microcurrent to flow through the solenoid 42 and controlling increase and decrease in current value while keeping the balance with the coil spring, the solenoid 42 can be moved to and held at an arbitrary angle. Note that, a structure including the solenoid drive circuit 310 and the like for operation of the piston 40 is an exemplary piston control unit for controlling operation of the piston.

Specifically, the piston is operated by the solenoid, and the piston control unit controls the piston through variable control of the drive current of the solenoid and ON/OFF control of the solenoid.

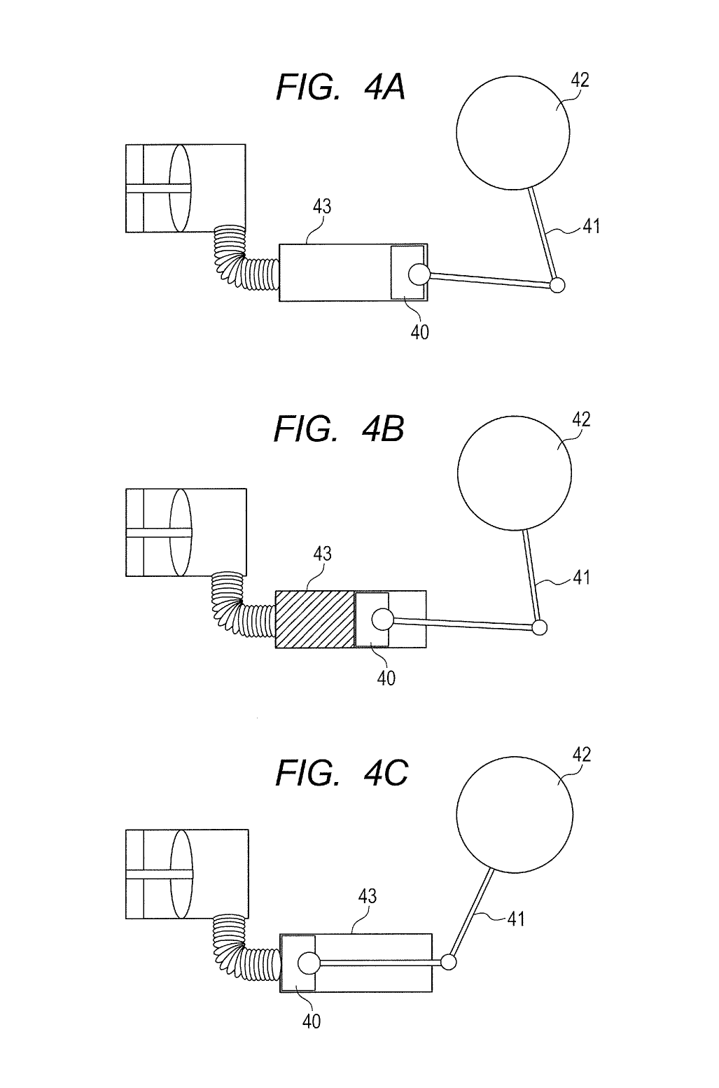

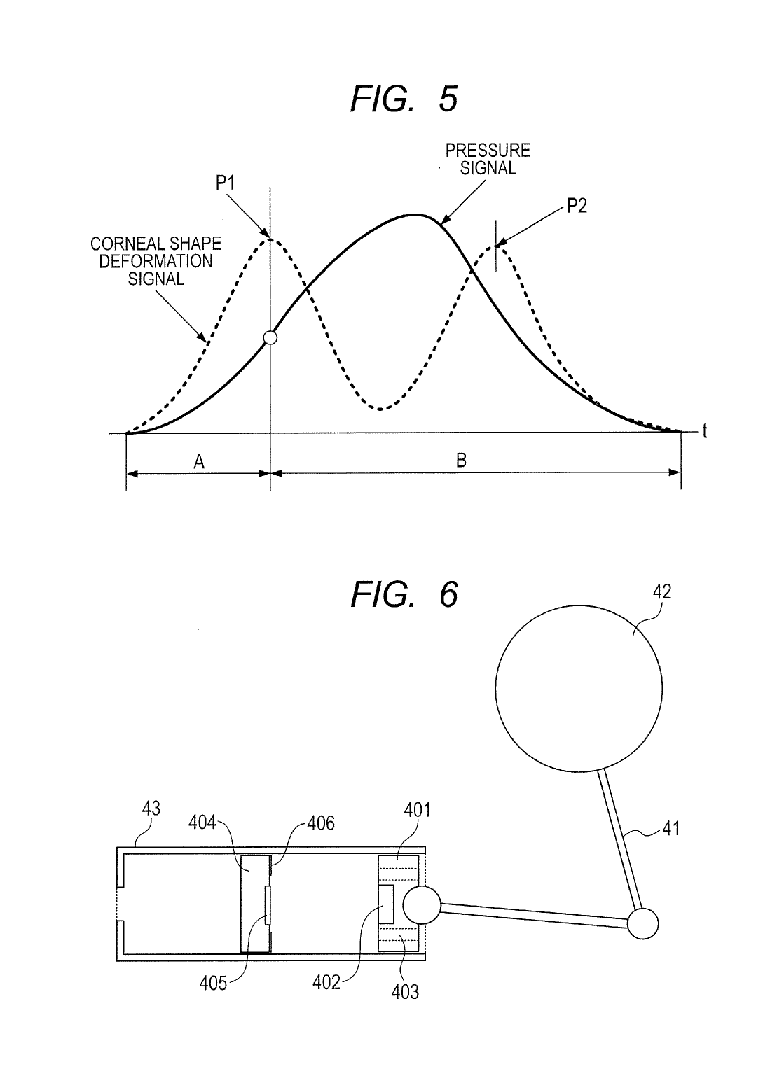

Prior to detailed, description of this embodiment, control of the solenoid by the system control portion 301 during related-art eye pressure measurement is described with reference to FIGS. 4A to 4C and FIG. 5. FIGS. 4A to 4C illustrate only an air discharge unit in the structural view of the optical system of FIG. 2. FIGS. 4A to 4C illustrate a state of energization of the solenoid 42 and the position of the piston 40 at that time. However, for the sake of simplicity of the description, the sensor dog 46 and the piston position detection switch 47 which are not necessary in the related art are omitted.

FIG. 5 shows a relationship between a pressure signal in the air chamber 23 obtained by the pressure sensor 45 and a state of deformation of the eye E to be inspected, which is detected by the light receiving element 34 (hereinafter referred to as corneal shape deformation signal), when the eye pressure is measured. In FIG. 5, a horizontal axis denotes a time from a start of the measurement while a vertical axis denotes levels of the respective signals.

Further, a time period A, in FIG. 5 is a time period during which the solenoid 42 is energized, and corresponds to a change in state from FIG. 4A to FIG. 4B, Similarly, a time period B in FIG. 5 is a time period during which a drive current of the solenoid 42 is stopped, and corresponds to a change in state from FIG. 4B to FIG. 4C.

FIG. 4A illustrates a piston position immediately before a start of energization of the solenoid 42. The piston 40 is fixed to a starting end of the cylinder, which is the initial position, by a torque of the coil spring which is built in the solenoid 42.

When the eye to be inspected and the tonometer are aligned and the eye pressure measurement is started, the system control portion 301 drives the solenoid 42 at high speed, and air in the air chamber 23 is pressurized by the piston 40 which is pushed forward by the solenoid 42. As the internal pressure of the air chamber 23 increases, air is discharged from the nozzle 22 toward the cornea Ec of the eye E to be inspected to start applanation of the cornea Ec. This time period is denoted as the time period A in FIG. 5.

As described above, the amount of light which enters the light receiving element 34 is designed to be at the maximum at the moment of the applanation of the cornea Ec by the discharged air, and a point P1 at which the corneal shape deformation signal is at the maximum in FIG. 5 is a moment at which the shape of the cornea Ec is changed from a convex shape to a concave shape.

When the maximum value of the corneal shape deformation signal is detected, the system control portion 301 stops the drive current of the solenoid 42, and, based on a value of the pressure signal shown as a hollow circle in FIG. 5 which is acquired at the same time, the system control portion 301 calculates the eye pressure value of the eye E to be inspected.

In general, it is said that an eye pressure value of a normal eye is 10 mmHg to 20 mmHg. It is known that, in the case of an eye disease such as glaucoma, the eye pressure value becomes as high as 20 mmHg or more. Therefore, the tonometer is required to have a wide measurement range from 0 mmHg to about 60 mmHg, and the volume of the cylinder 43 and the speed of the piston 40 are designed so that the maximum eye pressure value can be measured. In other words, it can be said that the volume of the cylinder of the tonometer is excessively large for an eye to be inspected having an ordinary eye pressure value which is smaller than the maximum eye pressure value.

Therefore, in the related-art measurement, control to reduce discharge of unnecessary air against an eye to be inspected is performed by reducing the drive current of the solenoid 42 and to advance the timing of stopping the drive current.

However, it is known that the piston 40 has an inertia force due to a mass of the piston 40 itself, and the piston 40 continues to move even after the drive current of the solenoid 42 is stopped.

FIG. 4B illustrates the position of the piston 40 at the moment of detection of the point P1 in FIG. 5, and FIG. 4C illustrates the position of the piston 40 when the piston 40 finally stops. Due to the inertia force of the piston 40, even after the drive current is stopped, the piston 40 maintains substantially the same speed, when moving from the position illustrated in FIG. 4B to the position illustrated in FIG. 4C, and pressurizes air hatched in FIG. 4B, which remains in the cylinder 43.

As a result, the pressurized air is discharged against the eye to be inspected as air unnecessary for the measurement. In the time period B shown in FIG. 5, the relationship between the corneal shape deformation signal and the pressure signal when the piston 40 is moved by the inertia force is shown.

It is known, that, even after the drive current of the solenoid 42 is stopped, the piston 40 continues to pressurize air in the cylinder 43 and the pressure in the air chamber 23 continues to increase. It can be seen that, as a result, air discharged from the nozzle 22 changes the shape of the cornea Ec from the applanation state to a concave shape, and thus, the corneal shape deformation signal value reduces.

After the piston 40 is in a stopped state as illustrated in FIG. 4C, due to the torque of the coil spring which is built in the solenoid 42, the piston 40 returns to the starting end of the cylinder which is the initial position illustrated in FIG. 4A.

Note that, through the stoppage of the discharge of air, the shape of the cornea Ec returns from the concave shape through the applanation state to the normal convex shape. In the process, the corneal shape deformation signal passes through a second peak point P2 as shown in FIG. 5.

Further, in this case, the timing of stopping the drive current of the solenoid 42 is not important, and thus, description is made on the assumption that the drive current is stopped after the maximum value of the corneal shape deformation signal is detected.

Detailed description is omitted, but, insofar as the peak value of the corneal shape deformation signal can be detected, the drive current may be stopped, for example, at the moment when the corneal shape deformation signal or the pressure signal exceeds a predetermined threshold value.

Again, in a related-art non-contact tonometer, the cylinder 43 is designed based on the maximum eye pressure, and thus, there is a problem in that air unnecessary for the measurement is discharged against the eye to be inspected due to the inertia force of the piston 40. Therefore, according to the present invention, the piston 40 has the separable structure including the air ejecting portion and the drive portion, and the above-mentioned problem is solved by changing the operation start position of the ejecting portion and changing (reducing) the initial volume of the cylinder 43.

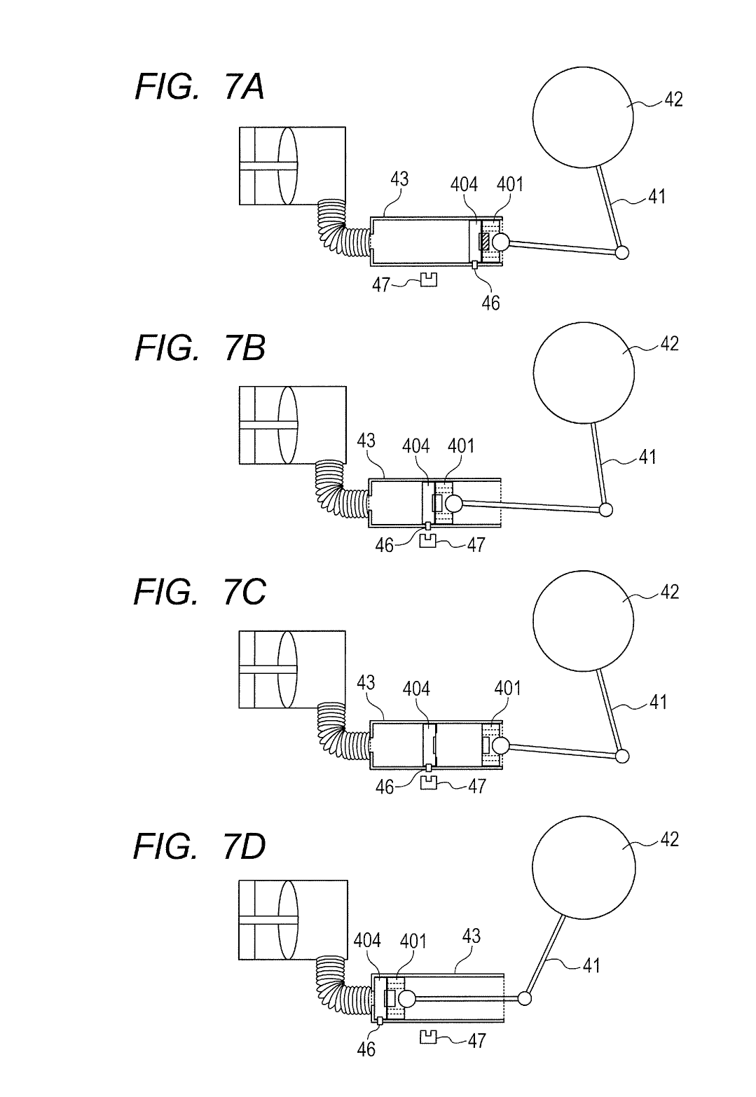

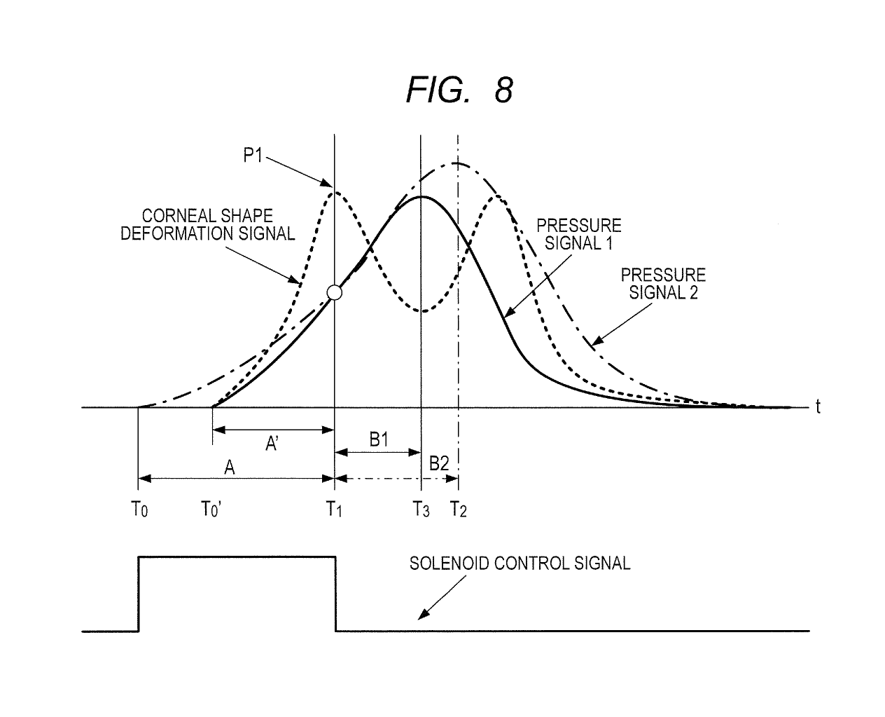

Next, the embodiment according to the present invention is described in detail, with reference to FIG. 6, FIGS. 7A to 7D, and FIG. 8.

FIG. 6 illustrates only the air discharge unit in the structural, view of the optical system of FIG. 2. The separable structure of the piston 40 is described.

In the first embodiment, the piston 40 is separated into a piston drive portion 401 and an air ejecting portion 404. The air ejecting portion 404 is provided in the cylinder 43 on an opening side on which an opening leading to the transfer tube 44 for sending air is provided. The piston drive portion 401 is connected to the above-mentioned piston control unit independently of the air ejecting portion 404. An electromagnet 402 whose ON/OFF control is performed from the outside is mounted to the piston drive portion 401. Further, the piston drive portion 401 has an air hole 403 for causing air between the piston drive portion 401 and the air ejecting portion 404 to escape. A metal or magnet 405 for substantially joining the drive portion 401 and the air ejecting portion 404 and a buffer 406 for lessening an impact sound when the drive portion 401 and the air ejecting portion 404 are substantially joined are mounted to the air ejecting portion 404. The air ejecting portion 404 can be stopped at an arbitrary position in the cylinder 43. However, in actual operation, as described below, the electromagnet 402 and the piston drive portion 401 may cause the air ejecting portion 404 to wait at a predetermined waiting position in the cylinder 43 which is set in accordance with the eye pressure.

Through use of a combination of an electromagnet and a magnet for substantially joining the piston drive portion 401 and the ejecting portion 404, the impact can be lessened by setting the same polarities for the electromagnet and the magnet in the measurement. Note that, in this embodiment, the above-mentioned position detecting unit detects the position of the air ejecting portion 404 in the cylinder 43.

FIGS. 7A to 7D are explanatory diagrams of operation of the air discharge unit illustrated in FIG. 6.

FIG. 8 shows a relationship between the pressure signal in the air chamber 23 obtained by the pressure sensor 45 and the corneal shape deformation signal which is detected, by the light receiving element 34 when the eye pressure is measured. The horizontal axis denotes a time elapsed from, the start of the measurement while the vertical axis denotes levels of the respective signals.

Similarly to the case of FIG. 5, a dotted line denotes the corneal shape deformation signal and a solid line denotes the pressure signal (pressure signal 1) according to the present invention. Further, for comparison purposes, a dot-and-dash line denotes the pressure signal (pressure signal 2) in the related-art control method (there is no corresponding corneal shape deformation signal).

FIG. 7A. illustrates a substantially joined state of the piston drive portion 401 and the air ejecting portion 404 according to the present invention. When the electromagnet 402 mounted to the piston drive portion 401 is in an ON state under the control of a solenoid electromagnet drive circuit 314, the piston drive portion 401 is joined to the ejecting portion 404. Further, the volume of the cylinder 43 can be arbitrarily set by moving, under the control of the solenoid drive circuit 310, the air ejecting portion 404 to the waiting position which is an arbitrary position as illustrated in FIG. 7B, and then, turning off the electromagnet 402 and returning the piston drive portion 401 to the initial position as illustrated in FIG. 7C.

In this case, the position of the air ejecting portion 404 is determined as the position detected by the above-mentioned sensor dog 46 and detection switch 47. Further, it suffices that the electromagnet 402 is in an ON state only when the ejecting portion 404 is moved in a direction away from the opening.

In this case, the positions of the detection sensor dog 46 and the detection switch 47 for detecting the operation start position of the piston 40 are set to be optimum positions at which the cylinder 43 has a volume necessary for measuring the eye pressure of an eye to be inspected that is 30 mmHg at the maximum.

When the measurement is started, similarly to the case of the related-art control, the solenoid 42 is driven at high speed during the time period A in FIG. 8, The solenoid 42 is driven and the piston drive portion 401 moves in the cylinder 43 at nigh speed. Then, from a time point at which the piston drive portion 401 and the air ejecting portion 404 are substantially joined (start of a time period A′), the pressure signal in the air chamber 23 increases, and applanation of the cornea Ec by air discharge from the nozzle 22 is started and the value of the corneal shape deformation signal also starts to increase.

When the eye pressure value of the eye to be inspected is smaller than the maximum eye pressure value set by the detection switch 47, the system control portion 301 detects the peak value P1 of the corneal shape deformation signal before the ejecting portion 404 which starts from the position illustrated in FIG. 7B reaches a terminating end of the cylinder 43 illustrated in FIG. 7D (FIG. 8).

When the peak value P1 of the corneal shape deformation signal is obtained, the system control portion 301 stops the drive current of the solenoid 42, and, based on a value of the pressure signal shown as a hollow circle in FIG. 8 which is acquired at the same time, the system control portion 301 calculates the eye pressure value of the eye E to be inspected.

As described with regard to the related-art control, even after the drive current of the solenoid 42 is stopped, the piston drive portion 401 continues to move to a position illustrated in FIG. 7D which is the terminating end of the cylinder 43 due to the inertia force.

However, the operation start position of the air ejecting portion 404 of the piston 40 is changed, and thus, a distance from the position illustrated in FIG. 7C to the position illustrated in FIG. 7D is smaller than that in the case of the related-art control. As a result, the amount of the discharged air is smaller. Further, a time period B1 shown in FIG. 8 from a time point when the peak of the corneal shape deformation signal is obtained to a time point when the pressure signal in the air chamber 23 is at the maximum, that is, a time period during which the piston 40 is moved due to the inertia force until the pressure signal in the air chamber 23 is at the maximum, is shorter than a time period B2 in the case of the related-art control from a time point, when the peak of the corneal shape deformation signal is obtained to a time point when the pressure signal in the air chamber 23 is at the maximum. Further, the solenoid 42 is driven from a time point which is before the air is pressurized, and thus, a leading edge of the pressure signal which is gentle in the case of the related-art control in the time period A shown in FIG. 8 can be set steeper as shown in the time period A1.

As described above, by changing the air pressurization start position and changing the initial volume of the cylinder 43 using the separable structure of the piston 40, unnecessary air discharge against the eye to be inspected is inhibited and an optimum amount of air can be discharged in accordance with the eye pressure value of the eye to be inspected. Note that, the above-mentioned various components of the piston 40 having the separable structure form a cylinder volume changing unit according to the present invention.

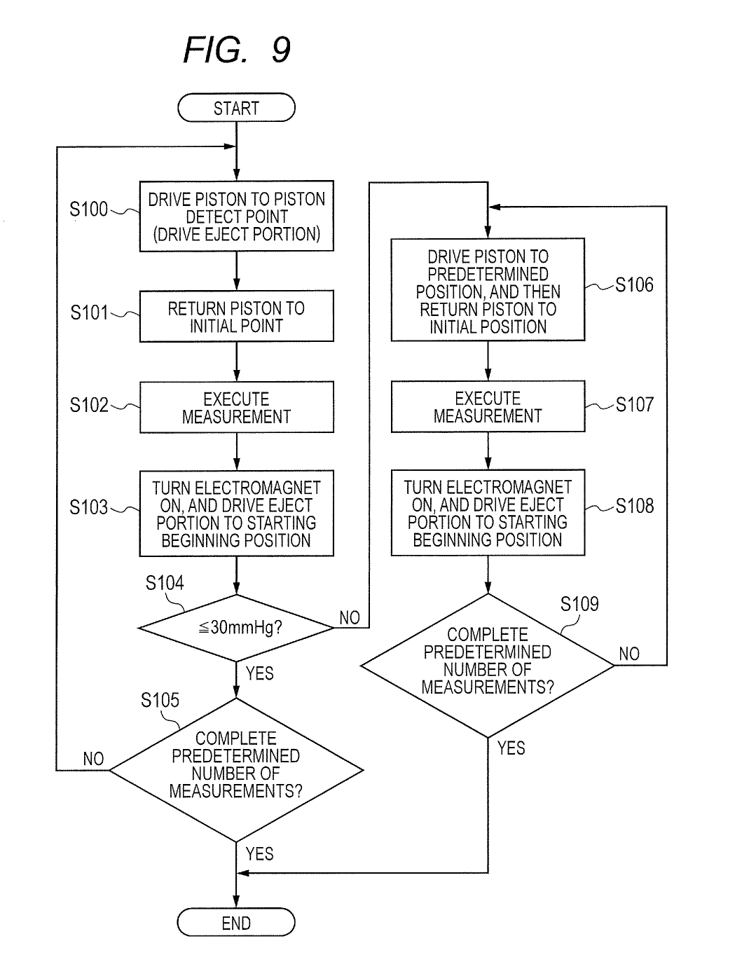

Finally, an exemplary embodiment of the present invention is described with reference to a flow chart of measurement of FIG. 9.

First, preparation before the start of measurement is described in brief. The examiner instructs the subject to rest his/her chin on the chin rest 112, and performs an adjustment with the chin rest motor 113 so that the eye to be inspected is at a predetermined height in the Y axis direction. The joystick 101 is operated until the screen reaches a position at which the corneal reflection image of the eye E to be inspected is displayed on the LCD monitor 116, and a measurement start button is depressed.

When the measurement start button is depressed, automatic alignment is started. In the alignment, a cornea bright spot imaged by the cornea Ec is split by the prism aperture 26, and is picked up by the CCD 28 together with the eye E to be inspected illuminated by the anterior ocular segment illumination light sources 30a and 30b and bright spot images of the anterior ocular segment illumination light sources 30a and 30b.

The system control portion 301 stores, in the memory 306, the anterior ocular segment image of the eye E to be inspected, which is picked up by the CCD 28, and, based on positional information of the respective bright spots extracted from the image of the eye E to be inspected and the corneal reflection image, alignment is performed via the motor drive circuit 312. After the alignment is completed, measurement in the following steps is started.

In Step S100, by causing a microcurrent to flow through the solenoid 42, the system control portion 301 drives the piston drive portion 401 at low speed, and moves the air ejecting portion 404 to the operation start position. The operation start position of the ejecting portion 404 is determined by the result of detection by the piston position detection switch 47.

It is assumed that, in this embodiment, the piston position detection switch 47 is set at a position at which a cylinder volume necessary for measuring the eye pressure of the eye to be inspected which is 30 mmHg at the maximum is secured.

When it is found, that the air ejecting portion 404 is moved to the operation start position, in Step S101, the system control portion 301 stops the current through the solenoid 42 to return the piston drive portion 401 to the initial position.

In Step S102, by driving the piston 40 at high speed, the eye pressure is measured. Then, in Step S103, the system control portion 301 again causes a current to flow through the solenoid 42 to move the piston drive portion 401 to the above-mentioned operating position, and then, stops the current through the solenoid 42 under a state in which the electromagnet 402 is ON, to thereby return the air ejecting portion to the initial position of the piston drive portion 401.

Then, in Step S104, it is determined whether or not the measured eye pressure value is equal to or smaller than 30 mmHg. In Step S100, the operation start position of the air ejecting portion 404 is changed, and thus, the tonometer according to this embodiment can measure the eye pressure of am eye to be inspected of only 30 mmHg at the maximum. Therefore, it is determined whether or not the measured eye pressure value is equal to or smaller than 30 mmHg. In the case of 30 mmHg or smaller, the process proceeds to Step S105. Note that, such a determination whether or not the measured value of an eye pressure is equal to or smaller than a predetermined value is executed by a module region which functions as a determination unit in the system control portion 301.

In Step S105, it is determined whether or not a predetermined number of measurements are completed. When the predetermined number of measurements are not completed, the process returns to Step S100 and the measurement is carried out again. When the predetermined number of measurements are completed, the eye pressure measurement ends. When the predetermined number of measurements is determined to be one, the measurement in Step S102 satisfies the condition, and thus, the eye pressure measurement ends.

Note that, when it is determined, as a result of the determination in Step S105, that further measurement is required to be carried out, the measurement is carried out again in Step S102, and then Step S104 may be omitted.

Next, control by the system control portion 301 when it is determined, in Step S104, that the result of the measurement is larger than 30 mmHg is described.

As described above, at the operation start position of the air ejecting portion 404 which is set in Step S100, an eye pressure which is larger than 30 mmHg cannot be measured. Therefore, the system control portion 301 proceeds to a measurement flow in which the operation in Step S100 is omitted.

Specifically, when the eye pressure value is larger than the predetermined, value, the cylinder volume changing unit increases the initial volume of the cylinder 43. After the predetermined operation start position of the air ejecting portion 404 is changed in Step S106, the measurement is started in Step S107. In other words, the waiting position of the air ejecting portion 404 is changed in accordance with the eye pressure of the eye to be inspected, which is measured by the eye pressure measurement unit, that is, the waiting position is shifted in the direction away from the above-mentioned opening.

Next, in Step S109, it is determined whether or not a predetermined number of measurements are completed. When the predetermined, number of measurements are not completed, tine process returns to Step S106 and the measurement is carried out again. When the predetermined number of measurements are completed in Step S109, the eye pressure measurement ends.

Note that, the flow may return to Step S100 when the result of the measurement is equal to or smaller than 30 mmHg.

After the eye pressure measurement ends in accordance with the above-mentioned flow chart, control is performed in accordance with an ordinary measurement routine involving switching between the right eye and the left eye and printing of the result of the measurement, and the entire operation ends.

Note that, according to this embodiment, the case of a single detection switch is described as an example, but there may be provided multiple detection switches of, for example, 15 mmHg, 30 mmHg, and 45 mmHg.

By carrying out the first eye pressure measurement under a state in which the cylinder volume corresponds to the eye pressure of 30 mmHg and, in the subsequent measurement, carrying out the measurement tinder a state in which the operation start position of the air ejecting portion 404 is set in accordance with the result of the first measurement, the measurement can be carried out under a state in which an optimum amount of air is discharged against the eye to be inspected.

For example, when the result of the first measurement is 10 mmHg, by setting the operation start position of the piston to be a position of the detection switch corresponding to the eye pressure of 15 mmHg, the measurement can be carried out with an amount of air which reduces discomfort of the subject.

Further, in this embodiment, the measurement is first carried out in the 30 mmHg mode, and then, as necessary, the measurement is carried out in the 60 mmHg mode. However, the measurement may be first carried out in the 60 mmHg mode, and, if the measured value is equal to or smaller than 30 mmHg, the measurement may then be carried out in the 30 mmHg mode.

Specifically, when the eye pressure value measured by the eye pressure measurement unit is equal to or smaller than a predetermined value, the initial volume of the cylinder for the air discharge is reduced by the cylinder volume changing unit.

Further, as a modification, an analog detecting unit such as a potentiometer or a rotary solenoid for detecting an angle may be used as the detection switch 47 instead of a digital detecting unit, with the result that more flexible control can be performed.

For example, by setting the operation start position of the piston for the second and later measurements to be a position at which “the result of the first measurement+5 mmHg” is the maximum eye pressure value which can be measured, a further optimum amount of air can be discharged against all eyes to be inspected. Specifically, by shifting the waiting position of the air ejecting portion 404 in a direction away from the opening in accordance with an eye pressure value obtained by adding a predetermined value to the measured eye pressure value, an optimum amount of air can be discharged.

In such a case, the cylinder volume changing unit changes the initial volume of the cylinder in accordance with an eye pressure value obtained by adding the predetermined value to the measured eye pressure value.

### Other Embodiments

Further, the present invention is also implemented by executing the following processing. Specifically, in this processing, software (program) for implementing the functions of the above-mentioned embodiment is supplied to a system or an apparatus via a network or various kinds of storage medium, and a computer (or CPU, MPU, or the like) of the system or the apparatus reads out and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application Mo. 2013-096401, filed May 1, 2013, which is hereby incorporated by reference herein in its entirety.

What is claimed is:

1. A non-contact tonometer, comprising:
a corneal shape deforming unit for pressurizing air in a cylinder using a piston provided in the cylinder and puffing the air through an opening in the cylinder toward a cornea, of an eye to be inspected so as to deform the cornea;
a piston control, unit for controlling operation of the piston; and
an eye pressure measurement unit for detecting a state of deformation, of the cornea so as to measure an eye pressure of the eye to be inspected,
the piston comprising:
an air ejecting portion provided on the opening side in the cylinder; and
a piston drive portion connected to the piston, control unit independently of the air ejecting portion.

2. A non-contact tonometer according to claim 1, wherein the air ejecting portion is configured to be joined to the piston drive portion, and to wait at a predetermined waiting position in the cylinder.

3. A non-contact tonometer according to claim 2, further comprising a position detecting unit for detecting a position of the air ejecting portion in the cylinder,
wherein the piston control unit moves the piston drive
portion so that the air ejecting portion is positioned at the predetermined waiting position based on a result of the detection by the position detecting unit.

4. A non-contact tonometer according to claim 1,
wherein the piston control unit comprises a solenoid, and
wherein the piston control unit controls the piston drive portion through at least one of variable control of a drive current of the solenoid and ON/OFF control, of the solenoid.

5. A non-contact tonometer according to claim 1, wherein the predetermined waiting position of the air ejecting portion is changed in accordance with a value of the eye pressure of the eye to be inspected, which is measured by the eye pressure measurement unit.

6. A non-contact tonometer according to claim 5, wherein the predetermined waiting position of the air ejecting portion is shifted away from the opening in the cylinder by the piston drive portion in accordance with the value of the eye pressure of the eye to be inspected, which is measured by the eye pressure measurement unit.

7. A non-contact tonometer according to claim 5, wherein the predetermined, waiting position of the air ejecting portion is shifted away from the opening in the cylinder by the piston drive portion in accordance with a value of the eye pressure obtained by adding a predetermined value to the value of the eye pressure of the eye to be inspected, which is measured by the eye pressure measurement unit.

8. A non-contact tonometer according to claim 5, further comprising a determination unit for determining whether or not the value of the eye pressure of the eye to be inspected, which is measured by the eye pressure measurement unit, is equal to or smaller than a predetermined value every time the eye pressure measurement unit carries out the measurement.

9. A non-contact tonometer according to claim 1, wherein the air ejecting portion and the piston drive portion are prevented from being joined when the piston drive portion returns to an operation start position in the measurement of the eye pressure.

10. A non-contact tonometer according to claim 1, further comprising a joining unit for joining the air ejecting portion and the piston drive portion,
the joining unit comprising an electromagnet for joining the air ejecting portion and the piston drive portion.
